(19) United States
(12) Patent Application Publication
(10) Pub. No.: US 2016/0318875 A1
(43) Pub. Date: Nov. 3, 2016

(54) PROCESSES AND INTERMEDIATES FOR THE PREPARATION OF ENZALUTAMIDE

(71) Applicant: SUN PHARMACEUTICAL INDUSTRIES LIMITED, Mumbai, Maharashtra (IN)

(72) Inventors: Ramendra Singh RATHORE, Farrukhabad (IN); Venugopal Venkatarama DURVASULA, Vizianagaram (IN); Amit SHARMA, Panchkula (IN); Ram Chander ARYAN, New Delhi (IN)

(21) Appl. No.: 15/105,127

(22) PCT Filed: Dec. 9, 2014
(86) PCT No.: PCT/IB2014/066735

§ 371 Date: Jun. 16, 2016

(30) Foreign Application Priority Data
Dec. 16, 2013 (IN) 3661/DEL/2013

(51) Int. Cl. C07D 233/86; C07D 207/408; C07D 249/18; C07C 333/08; C07C 231/12

(52) U.S. Cl. CPC C07D 233/86; C07C 333/08; C07C 231/12; C07D 249/18; C07D 207/408

(57) ABSTRACT

The present invention provides processes for the preparation of enzalutamide.

### FIELD OF THE INVENTION

The present invention provides processes for the preparation of enzalutamide.

### BACKGROUND OF THE INVENTION

Enzalutamide is chemically described as 4-{3-[4-cyano-3-(trifluoromethyl)phenyl]-5,5-dimethyl-4-oxo-2-sulfanylideneimidazolidin-1-yl}-2-fluoro-N-methylbenzamide, and is depicted in Formula I.

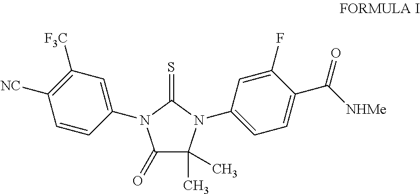 [SMILES: CNC(=O)c1ccc(N2C(=S)N(c3ccc(C#N)c(C(F)(F)F)c3)C(=O)C2(C)C)cc1F]

Processes for the preparation of enzalutamide are described in U.S. Publication Nos. 2007/0004753 and 2007/0254933; and PCT Publication Nos. WO 2007/127010, WO 2006/124118, and WO 2011/106570.

PCT Publication No. WO 2011/106570 discloses that the processes described in U.S. Publication Nos. 2007/0004753 and 2007/0254933 result in only a 25% yield of enzalutamide in the final step, which accounts for a 15% overall yield. PCT Publication No. WO 2011/106570 further discloses that the known processes for preparing enzalutamide involve the use of extremely toxic reagents, for example, acetone cyanohydrin.

It is acknowledged in WO 2011/106570 [Para 0010] and also a well known fact that Acetone cyanohydrin is toxic and therefore its use as a reagent should be avoided for the industrial production of a pharmaceutical ingredient. Therefore, there is a need in the art to develop a process for the preparation of enzalutamide that avoids the use of acetone cyanohydrin as a reagent.

### SUMMARY OF THE INVENTION

The present invention provides a process for the preparation of enzalutamide that does not involve the use of toxic reagents and, at the same time, results in a higher yield of enzalutamide.

### DETAILED DESCRIPTION OF THE INVENTION

The term “about,” as used herein, refers to any value which lies within the range defined by a number up to ±10% of the value.

A first aspect of the present invention provides a process for the preparation of enzalutamide of Formula I,

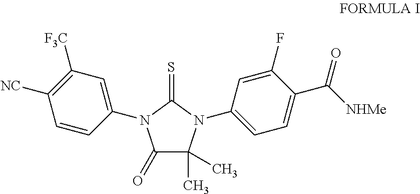 [SMILES: CNC(=O)c1ccc(N2C(=S)N(c3ccc(C#N)c(C(F)(F)F)c3)C(=O)C2(C)C)cc1F]

which comprises:
a) reacting a compound of Formula II

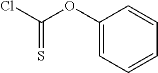

with a compound of Formula III

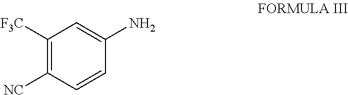 [SMILES: N#Cc1ccc(N)cc1C(F)(F)F]

to prepare a compound of Formula IV; and

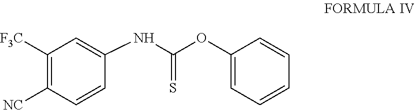 [SMILES: N#Cc1ccc(NC(=S)Oc2ccccc2)cc1C(F)(F)F]

b) reacting the thus obtained compound of Formula IV obtained in above step a) with a compound of Formula V,

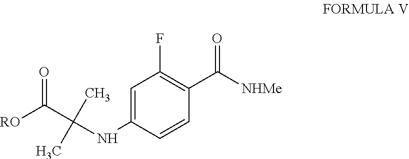

wherein R is methyl, ethyl, benzyl,

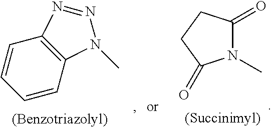

A second aspect of the present invention provides a process for the preparation of enzalutamide of Formula I,

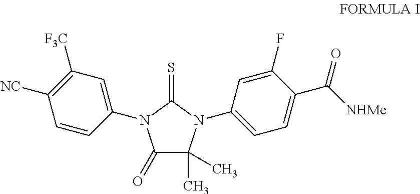 [SMILES: CNC(=O)c1ccc(N2C(=S)N(c3ccc(C#N)c(C(F)(F)F)c3)C(=O)C2(C)C)cc1F]

which comprises reacting a compound of Formula IV with

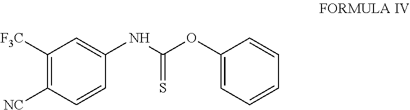 [SMILES: N#Cc1ccc(NC(=S)Oc2ccccc2)cc1C(F)(F)F]

a compound of Formula V,

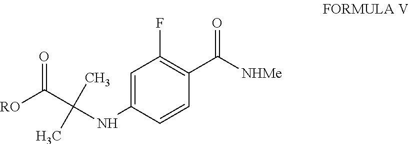

wherein R is methyl, ethyl, benzyl,

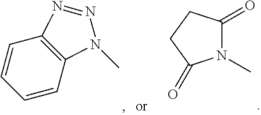

The reaction of the compound of Formula IV with the compound of Formula V is carried out in a solvent. The solvent is selected from the group consisting of water, dimethyl sulfoxide, esters, ethers, alcohols, hydrocarbons, halogenated hydrocarbons, and mixtures thereof. Examples of preferred ester solvents include ethyl acetate, butyl acetate, and isopropyl acetate. Examples of preferred alcohol solvents include methanol, ethanol, and n-butanol. Examples of preferred hydrocarbon solvents include hexane and heptane. Examples of preferred ether solvents include tetrahydrofuran and diisopropyl ether. An example of a preferred halogenated hydrocarbon is dichloromethane.

The reaction of the compound of Formula IV with the compound of Formula V is carried out for about 15 hours to about 20 hours, preferably about 15 hours to about 19 hours.

The reaction of the compound of Formula IV with the compound of Formula V is carried out at about 60° C. to about 100° C., preferably about 70° C. to about 90° C.

The compound of Formula I obtained may be isolated by employing one or more techniques selected from the group consisting of filtration, decantation, extraction, distillation, evaporation, chromatography, precipitation, concentration, crystallization, centrifugation, or recrystallization, and may further be dried using conventional techniques, for example, drying, drying under vacuum, spray drying, freeze drying, air drying, or agitated thin film drying.

A third aspect of the present invention provides a process for the preparation of a compound of Formula IV,

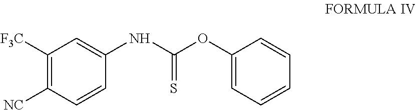 [SMILES: N#Cc1ccc(NC(=S)Oc2ccccc2)cc1C(F)(F)F]

which comprises reacting a compound of Formula II

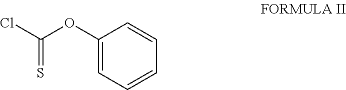

with a compound of Formula III.

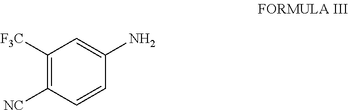 [SMILES: N#Cc1ccc(N)cc1C(F)(F)F]

The compound of Formula III can be prepared by the methods known in the art, for example, PCT Publication Nos. WO 2007/127010 and WO 2006/124118. The compound of Formula II can be prepared by the method disclosed in U.S. Pat. No. 4,754,072 or by the method as described herein.

The reaction of the compound of Formula II with the compound of Formula III to give the compound of Formula IV is carried out in a solvent.

The solvent is selected from the group consisting of water, dimethyl sulfoxide, esters, ethers, alcohols, hydrocarbons, halogenated hydrocarbons, amides, and mixtures thereof. Examples of preferred ester solvents include ethyl acetate, butyl acetate, and isopropyl acetate. Examples of preferred alcohol solvents include methanol, ethanol, and n-butanol. Examples of preferred hydrocarbon solvents include hexane and heptane. Examples of preferred ether solvents include tetrahydrofuran and diisopropyl ether. An example of a preferred halogenated hydrocarbon is dichloromethane Examples of preferred amide solvents include N,N-dimethyl formamide and acetamide.

The reaction of the compound of Formula II with the compound of Formula III is carried out for about 15 hours to about 25 hours, preferably, about 16 hours to about 24 hours.

The reaction of the compound of Formula II with the compound of Formula III is carried out at about 10° C. to about 40° C., preferably, about 15° C. to about 30° C.

The compound of Formula IV obtained by the reaction of the compound of Formula II with the compound of Formula III may optionally be isolated by employing one or more techniques selected from the group consisting of filtration, decantation, extraction, distillation, evaporation, chromatography, precipitation, concentration, crystallization, centrifugation, or recrystallization, and may further be dried using conventional techniques, for example, drying, drying under vacuum, spray drying, freeze drying, air drying, or agitated thin film drying.

A fourth aspect of the present invention provides a process for the preparation of a compound of Formula V,

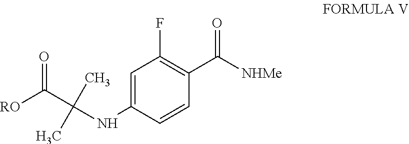

which comprises reacting a compound of Formula VI or a salt thereof

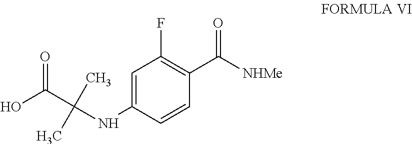 [SMILES: CNC(=O)c1ccc(NC(C)(C)C(=O)O)cc1F]

with a compound R—OH,
wherein R is methyl, ethyl, benzyl,

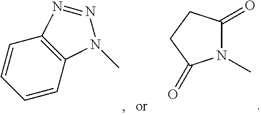

A fifth aspect of the present invention provides a process for the preparation of a compound of Formula V,

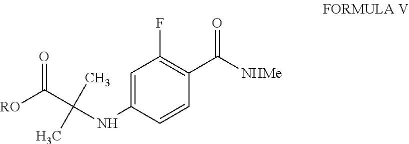

which comprises reacting a compound of Formula VI or a salt thereof

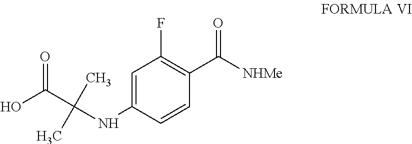 [SMILES: CNC(=O)c1ccc(NC(C)(C)C(=O)O)cc1F]

with a compound R—OH,
wherein R is

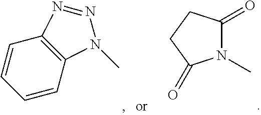

The compound of Formula VI can be prepared by the methods known in art, for example, PCT Publication No. WO 2011/106570.

The reaction of the compound of Formula VI with the compound R—OH to give the compound of Formula V can be carried out in the presence of N,N-dimethylamino pyridine.

The reaction of the compound of Formula VI with the compound R—OH to give the compound of Formula V is carried out in the optional presence of a coupling agent in a solvent.

The coupling agent can be selected from the group consisting of 1-ethyl-3-(3-dimethylaminopropyl)carbodiimide hydrochloride, N,N′-dicyclohexylcarbodiimide, thionyl chloride, and oxalyl chloride.

The solvent is selected from the group consisting of water, esters, halogenated hydrocarbons, ethers, alcohols, hydrocarbons, amides, and mixtures thereof. Examples of preferred ester solvents include ethyl acetate, butyl acetate, and isopropyl acetate. An example of a preferred halogenated hydrocarbon is dichloromethane Examples of preferred alcohol solvents include methanol, ethanol, and n-butanol. Examples of preferred hydrocarbon solvents include hexane and heptane. Examples of preferred ether solvents include tetrahydrofuran and diisopropyl ether. Examples of preferred amide solvents include N,N-dimethyl formamide and acetamide.

The reaction of the compound of Formula VI with the compound R—OH is carried out for about 2 hours to about 8 hours, preferrably about 3 hours to about 6 hours.

The reaction of the compound of Formula VI with the compound R—OH is carried out at about 5° C. to about 30° C., preferrably about 10° C. to about 30° C.

The compound of Formula V may optionally be isolated by employing one or more techniques selected from the group consisting of filtration, decantation, extraction, distillation, evaporation, chromatography, precipitation, concentration, crystallization, centrifugation, or recrystallization, and may further be dried using conventional techniques, for example, drying, drying under vacuum, spray drying, freeze drying, air drying, or agitated thin film drying.

A sixth aspect of the present invention provides a compound of Formula IV.

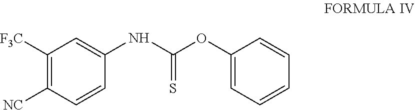 [SMILES: N#Cc1ccc(NC(=S)Oc2ccccc2)cc1C(F)(F)F]

A seventh aspect of the present invention provides a compound of Formula V,

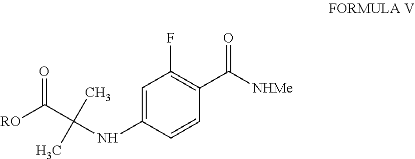

wherein R is

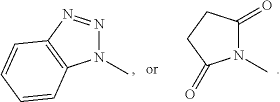

An eighth aspect of the present invention provides the use of a compound of Formula IV for the preparation of enzalutamide.

A ninth aspect of the present invention provides the use of a compound of Formula V for the preparation of enzalutamide.

A tenth aspect of the present invention provides a process for the preparation of enzalutamide of Formula I,

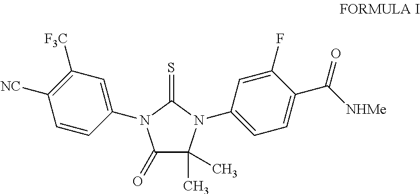 [SMILES: CNC(=O)c1ccc(N2C(=S)N(c3ccc(C#N)c(C(F)(F)F)c3)C(=O)C2(C)C)cc1F]

which comprises reacting a compound of Formula V

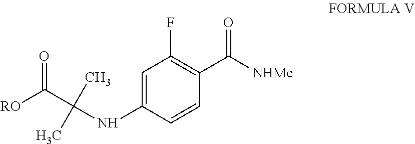

with a compound of Formula VII,

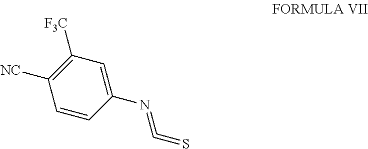 [SMILES: N#Cc1ccc(N=C=S)cc1C(F)(F)F]

wherein R is

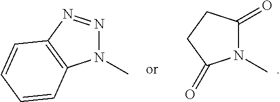

The compound of Formula VII can be prepared by the methods known in the art, for example, PCT Publication Nos. WO 2007/127010 and WO 2006/124118.

The reaction of the compound of Formula V with the compound of Formula VII is carried out in a solvent. The solvent is selected from the group consisting of water, dimethyl sulfoxide, esters, ethers, alcohols, hydrocarbons, halogenated hydrocarbons, and mixtures thereof. Examples of preferred ester solvents include ethyl acetate, butyl acetate, and isopropyl acetate. Examples of preferred alcohol solvents include methanol, ethanol, and n-butanol. Examples of preferred hydrocarbon solvents include hexane and heptane. Examples of ether solvents include tetrahydrofuran and diisopropyl ether. An example of a preferred halogenated hydrocarbon is dichloromethane.

The reaction of the compound of Formula V with the compound of Formula VII is carried out for about 10 hours to about 18 hours, preferably, about 12 hours to about 16 hours.

The reaction of the compound of Formula V with the compound of Formula VII is carried out at about 60° C. to about 100° C., preferably, about 70° C. to about 90° C.

The compound of Formula I may be isolated by employing one or more techniques selected from the group consisting of filtration, decantation, extraction, distillation, evaporation, chromatography, precipitation, concentration, crystallization, centrifugation, or recrystallization, and may further be dried using conventional techniques, for example, drying, drying under vacuum, spray drying, freeze drying, air drying, or agitated thin film drying.

An embodiment of the present invention provides a process according to the first, second and tenth aspects, wherein enzalutamide obtained is free from the compounds of Formula IV and Formula V.

An embodiment of the present invention provides a process according to the first, second and tenth aspects, wherein enzalutamide obtained is having less than 0.5% of the compound of Formula IV.

An embodiment of the present invention provides a process according to the first, second and tenth aspects, wherein enzalutamide obtained is having less than 0.5% of the compound of Formula V.

An eleventh aspect of the present invention provides the use of enzalutamide free from the compounds of Formula IV and Formula V for the manufacturing of a medicament used for the treatment of metastatic castration-resistant prostate cancer.

A twelfth aspect of the present invention provides the use of enzalutamide having less than 0.5% of the compound of Formula IV for the manufacturing of a medicament used for the treatment of metastatic castration-resistant prostate cancer.

A thirteenth aspect of the present invention provides the use of enzalutamide having less than 0.5% of the compound of Formula V for the manufacturing of a medicament used for the treatment of metastatic castration-resistant prostate cancer.

A fourteenth aspect of the present invention provides the use of enzalutamide free from the compounds of Formula IV and Formula V for the preparation of a pharmaceutical composition.

### Methods:

The IR spectrum was recorded using a PerkinElmer Spectrum One FTIR spectrometer.

The Mass spectrum was recorded using an Ab Sciex® API 2000 LC/MS/MS system.

The NMR spectrum was recorded using a Bruker® Avance III 400 MHz NMR spectrometer.

While the present invention has been described in terms of its specific aspects, certain modifications and equivalents will be apparent to those skilled in the art and are intended to be included within the scope of the present invention.

### EXAMPLES

### Example 1

### Process for the preparation of O-phenyl carbonochloridothioate (Formula II)

A solution of 5% aqueous sodium hydroxide (14.28 g) in phenol (30 g) was added to a cooled solution of thiophosgene (24.3 mL) over 2 hours at 0° C. to 5° C. The reaction mixture was stirred for 1 hour to 2 hours at 0° C. to 5° C. The layers obtained were separated, and then the organic layer obtained was concentrated at 40° C. to obtain the title compound.

Yield: 37.1 g.

### Example 2

### Process for the preparation of O-phenyl [4-cyano-3-(trifluoromethyl)phenyl]carbamothioate (Formula IV)

4-Amino-2-(trifluoromethyl)benzonitrile (Formula III; 20 g) was added to dichloromethane (200 mL) and a solution of O-phenyl carbonochloridothioate (Formula II; 10 g) in dichloromethane (100 mL) was added to the reaction mixture over 10 minutes at 20° C. to 25° C. N,N-dimethyl formamide (20 mL) was added to the reaction mixture and the reaction mixture was stirred for 18 hours at 20° C. to 25° C. The reaction mixture was cooled to 0° C. to 5° C. and the solid obtained was filtered at 0° C. to 5° C. The filtrate obtained was concentrated at 40° C. under vacuum and a solid material was obtained. A mixture of hexanes (1 L) and hydrochloric acid (1 L) was added to the solid material, and then the reaction mixture was stirred for 20 minutes. The solid obtained was filtered, and then dried under vacuum at 40° C. to obtain the title compound.

Yield: 12.8 g.

¹H NMR (400 MHz, CDCl₃), δ (in ppm): 8.7 (s, 1H), 8.05 (brs, 2H), 7.84 (d, j=8.36 Hz, 1H), 7.3-7.5 (m, 3H), 7.13 (d, j=7.8 Hz, 2H).

Mass: [M+H]⁺=322.9; MS/MS: 322.9, 111.1, 94.7.

IR in KBr, (in cm⁻¹): 3433, 3196, 3159, 3039, 2232, 1707, 1613, 1592, 1538, 1504, 1490, 1456, 1411, 1424, 1370, 1323, 1308, 1277, 1220, 1149, 1196, 1173, 1149, 1136, 1070, 1051, 1022, 1003, 940, 903, 841, 774, 752, 734, 691, 677, 638, 628, 613, 554, 528, 493, 452.

### Example 3

### Process for the preparation of O-phenyl [4-cyano-3-(trifluoromethyl)phenyl]carbamothioate (Formula IV)

4-Amino-2-(trifluoromethyl)benzonitrile (Formula III; 0.5 g) was added to dichloromethane (5 mL). N,N-dimethyl formamide (1 mL) was added to the reaction mixture and the reaction mixture was stirred for 5 minutes. O-Phenyl carbonochloridothioate (Formula II; 0.508 g) and neutral alumina (1 g) at 20° C. to 25° C. were added to the reaction mixture, and then the reaction mixture was stirred for 24 hours. The solid obtained was filtered, and then washed with dichloromethane (10 mL). The filtrate was washed with water (10 mL) for 5 minutes and the layers were separated. The organic layer was dried with sodium sulfate, and then filtered. The solution obtained was concentrated to yield a solid material, which was crystallized using a dichloromethane and hexanes mixture to obtain the title compound.

Yield: 0.52 g.

### Example 4

### Process for the preparation of 1H-benzotriazol-1-yl N-[3-fluoro-4-(methyl carbamoyl)phenyl]-2-methylalaninate (Formula V; when R=benzotriazolyl)

N-[3-fluoro-4-(methylcarbamoyl)phenyl]-2-methylalanine hydrochloride (a salt of Formula VI; 1 g) was added to dichloromethane (10 mL) followed by the addition of 1H-benzotriazol (0.52 g) and 1-ethyl-3-(3-dimethylaminopropyl)carbodiimide hydrochloride (0.64 g) at 20° C. to 25° C. The reaction mixture was heated at 20° C. to 25° C. for 5 hours to 6 hours. Water (10 mL) was added to the reaction mixture, and then the reaction mixture was stirred for 30 minutes. The layers obtained were separated and the organic layer was concentrated to obtain the title compound.

Yield: 1.4 g.

¹H NMR (400 MHz, CDCl₃), δ (in ppm): 8.03 (m, 2H), 7.4 (m, 2H), 6.98 (d, 1H, j=8.24 Hz), 6.67 (brs, 1H), 6.58 (dd, 1H), 6.41 (dd, 1H), 3.03 (d, 1H), 1.88 (s, 1H).

Mass: [M+H]⁺=372.4; MS/MS: 372.3, 344.2, 209.1.

The compounds of Formula V (when R=methyl, ethyl, or benzyl) can be prepared by the method disclosed in Example 4 by replacing 1H-benzotriazol with the compound of Formula R—OH (when R=methyl, ethyl, or benzyl).

### Example 5

### Process for the preparation of 2,5-dioxopyrrolidin-1-yl-N-[3-fluoro-4-(methylcarbamoyl)phenyl]-2-methylalaninate (Formula V; when R=succinimyl)

N-[3-fluoro-4-(methylcarbamoyl)phenyl]-2-methylalanine hydrochloride (a salt of Formula VI; 500 mg) was added to ethyl acetate (10 mL) at 24° C. The reaction mixture was stirred and cooled to 0° C. over 10 minutes. N,N′-dicyclohexylcarbodiimide (426 mg) was added to the reaction mixture at 0° C., followed by the addition of N,N-dimethylaminopyridine (24 mg), and N-hydroxy succinamide (249 mg). The reaction mixture was stirred for 5 minutes, and then the temperature was increased to 15° C. The reaction mixture was stirred at 12° C. to 18° C. for 3 hours. Water (10 mL) was added to the reaction mixture at 15° C. to 20° C., and then the reaction mixture was stirred for 5 minutes. The reaction mixture was filtered through celite, and then washed with ethyl acetate (10 mL). The layers obtained were separated, and then the organic layer was washed with sodium bicarbonate solution (7%) at 18° C. to 20° C. The layers obtained were separated, and then the organic layer was dried over sodium sulphate and filtered. The organic layer was further distilled under vacuum at 18° C. to 20° C. for 1 hour to obtain the title compound.

Yield: 625 mg.

¹H NMR (400 MHz, CDCl₃), δ (in ppm): 7.69 (m, 1H), 7.48 (t, 1H), 6.45 (q, 1H), 6.26 (q, 1H), 2.8 (m, 4H), 2.74 (d, 3H, j=4.52 Hz), 1.64 (s, 6H).

Mass: [M+H]⁺=352.1, 321.2, 237, 209.1, 195.1, 178, 169.1, 152.1, 138.1, 133.1, 111.9, 58.2.

### Example 6

### Process for the Preparation of Enzalutamide

Ethyl N-[3-Fluoro-4-(methylcarbamoyl)phenyl]-2-methylalaninate (Formula V when R=ethyl; 0.5 g), dimethyl sulfoxide (0.5 mL), and O-phenyl [4-cyano-3-(trifluoromethyl)phenyl]carbamothioate (Formula IV; 2.26 g) were added to isopropyl acetate (1 mL). The reaction mixture was heated to 80° C. to 85° C., and then stirred for 18 hours. Dichloromethane (20 mL) and water (20 mL) were added to the reaction mixture, then the mixture was stirred for 15 minutes. The layers obtained were separated, and then the organic layer was concentrated at 25° C. under vacuum to obtain the title compound.

Yield: 2.5 g

Enzalutamide can also be prepared by reacting O-phenyl [4-cyano-3-(trifluoromethyl)phenyl]carbamothioate (Formula IV) with the compound of Formula V (when R=methyl or benzyl) by following the method disclosed in Example 6.

### Example 7

### Process for the Preparation of Enzalutamide

1H-benzotriazol-1-yl-N-[3-fluoro-4-(methylcarbamoyl)phenyl]-2-methylalaninate (Formula V, when R=benzotriazolyl; 0.5 g), dimethyl sulfoxide (0.5 mL), and O-phenyl [4-cyano-3-(trifluoromethyl)phenyl]carbamothioate (Formula IV; 0.87 g) were added to isopropyl acetate (1 mL). The reaction mixture was heated to 80° C. to 85° C., and then stirred for 16 hours. Dichloromethane (20 mL) and water (20 mL) were added to the reaction mixture, and then the mixture was stirred for 15 minutes. The reaction mixture was filtered through celite, and then washed with dichloromethane (10 mL). The layers obtained were separated, and then the organic layer was concentrated at 25° C. under vacuum to obtain the title compound.

Yield: 0.61 g

### Example 8

### Process for the Preparation of Enzalutamide

Enzalutamide can also be prepared by the method disclosed in Example 7 by replacing 1H-benzotriazol-1-yl N-[3-fluoro-4-(methylcarbamoyl)phenyl]-2-methylalaninate with 2,5-dioxopyrrolidin-1-yl-N-[3-fluoro-4-(methylcarbamoyl)phenyl]-2-methylalaninate.

### Example 9

### Process for the Preparation of Enzalutamide

1H-benzotriazol-1-yl-N-[3-fluoro-4-(methylcarbamoyl)phenyl]-2-methylalaninate (Formula V when R=benzotriazolyl; 0.5 g) and 4-isothiocyanato-2-(trifluoromethyl)-benzonitrile (Formula VII; 0.62 g) were added to a mixture of dimethyl sulfoxide (0.5 mL) and isopropyl acetate (1 mL). The reaction mixture was heated to 80° C. to 85° C., and then stirred for 16 hours. Dichloromethane (20 mL) and water (20 mL) were added to the reaction mixture, and then the mixture was stirred for 15 minutes. The reaction mixture was filtered through celite, and then washed with dichloromethane (10 mL). The layers obtained were separated, and then the organic layer was concentrated at 25° C. under vacuum to obtain the title compound.

Yield: 1.1 g.

### Example 10

### Process for the Preparation of Enzalutamide

Enzalutamide can also be prepared by reacting 4-isothiocyanato-2-(trifluoromethyl)-benzonitrile with 2,5-dioxopyrrolidin-1-yl-N-[3-fluoro-4-(methylcarbamoyl)phenyl]-2-methylalaninate by following the method disclosed in Example 9.

1. A process for the preparation of enzalutamide of Formula I, 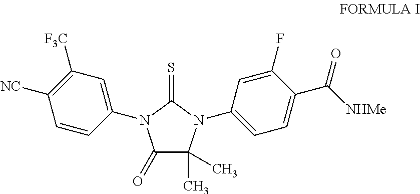 [SMILES: CNC(=O)c1ccc(N2C(=S)N(c3ccc(C#N)c(C(F)(F)F)c3)C(=O)C2(C)C)cc1F]
which comprises:
a) reacting a compound of Formula II 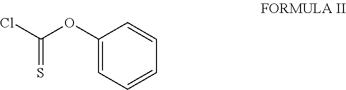
with a compound of Formula III 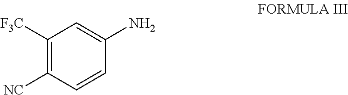 [SMILES: N#Cc1ccc(N)cc1C(F)(F)F]
to obtain a compound of Formula IV; 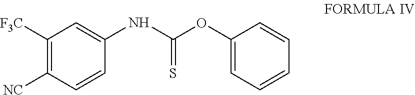 [SMILES: N#Cc1ccc(NC(=S)Oc2ccccc2)cc1C(F)(F)F]
and
b) reacting the thus obtained compound of Formula IV obtained in above step a) with a compound of Formula V, 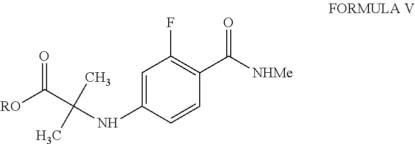
wherein R is methyl, ethyl, benzyl,

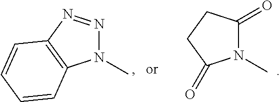

2. A process for the preparation of enzalutamide of Formula I, 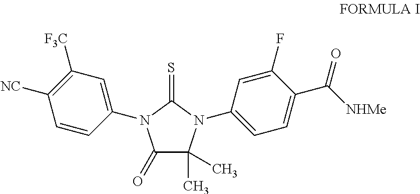 [SMILES: CNC(=O)c1ccc(N2C(=S)N(c3ccc(C#N)c(C(F)(F)F)c3)C(=O)C2(C)C)cc1F]
which comprises reacting a compound of Formula IV with 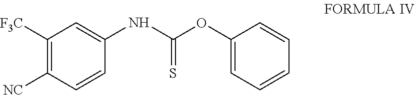 [SMILES: N#Cc1ccc(NC(=S)Oc2ccccc2)cc1C(F)(F)F]
a compound of Formula V, 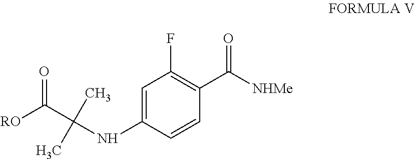
wherein R is methyl, ethyl, benzyl,

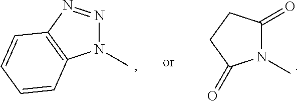

3. A process for the preparation of a compound of Formula IV, 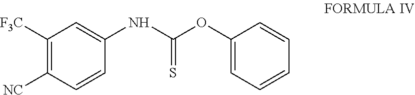 [SMILES: N#Cc1ccc(NC(=S)Oc2ccccc2)cc1C(F)(F)F]
which comprises reacting a compound of Formula II 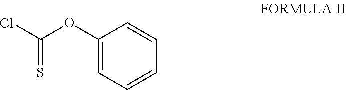
with a compound of Formula III.

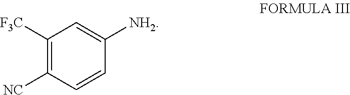 [SMILES: N#Cc1ccc(N)cc1C(F)(F)F]

4. The process according to claim 1 or claim 3, wherein the reaction of the compound of Formula II with the compound of Formula III to give the compound of Formula IV is carried out in a solvent selected from the group consisting of water, dimethyl sulfoxide, esters, ethers, alcohols, hydrocarbons, halogenated hydrocarbons, amides, and mixtures thereof.

5. The process according to claim 1 or claim 2, wherein the reaction of the compound of Formula IV with the compound of Formula V is carried out in a solvent selected from the group consisting of water, dimethyl sulfoxide, esters, ethers, alcohols, hydrocarbons, halogenated hydrocarbons, and mixtures thereof.

6. A process for the preparation of a compound of Formula V, 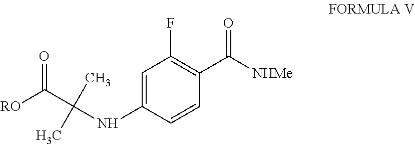
which comprises reacting a compound of Formula VI or a salt thereof 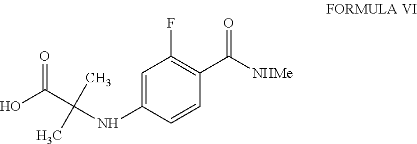 [SMILES: CNC(=O)c1ccc(NC(C)(C)C(=O)O)cc1F]
with a compound R—OH,
wherein R is methyl, ethyl, benzyl,

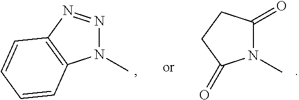

7. (canceled)

8. The process according to claim 6, wherein the reaction of the compound of Formula VI with the compound R—OH to give the compound of Formula V is carried out in the presence of a coupling agent selected from the group consisting of 1-ethyl-3-(3-dimethylaminopropyl)carbodiimide hydrochloride, N,N′-dicyclohexylcarbodiimide, thionyl chloride, and oxalyl chloride.

9. The process according to claim 6, wherein the reaction of the compound of Formula VI with the compound R—OH to give the compound of Formula V is carried out in the presence of a solvent selected from the group consisting of water, esters, halogenated hydrocarbons, ethers, alcohols, hydrocarbons, amides, and mixtures thereof.

10. The process according to claim 6, wherein the reaction of the compound of Formula VI with the compound R—OH to give the compound of Formula V is carried out in the presence of N,N-dimethylaminopyridine.

11. A compound of Formula IV.

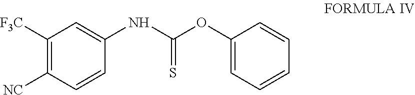 [SMILES: N#Cc1ccc(NC(=S)Oc2ccccc2)cc1C(F)(F)F]

12. A compound of Formula V, 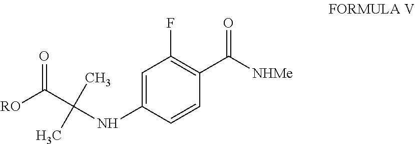
wherein R is

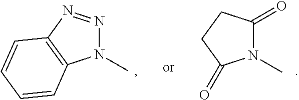

13. Use of a compound of Formula IV for the preparation of enzalutamide.

14. Use of a compound of Formula V for the preparation of enzalutamide.

15. A process for the preparation of enzalutamide of Formula I, 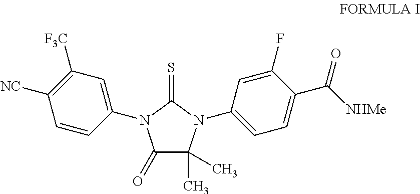 [SMILES: CNC(=O)c1ccc(N2C(=S)N(c3ccc(C#N)c(C(F)(F)F)c3)C(=O)C2(C)C)cc1F]
which comprises reacting a compound of Formula V 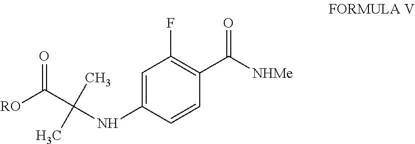
with a compound of Formula VII, 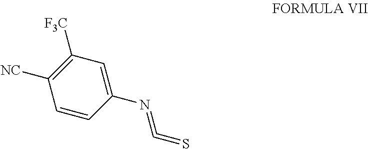 [SMILES: N#Cc1ccc(N=C=S)cc1C(F)(F)F]
wherein R is

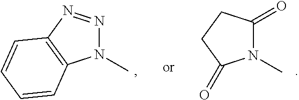

16. The process according to claim 14, wherein the reaction of the compound of Formula V with the compound of Formula VII is carried out in a solvent selected from the group consisting of water, dimethyl sulfoxide, esters, ethers, alcohols, hydrocarbons, halogenated hydrocarbons, and mixtures thereof.

17-23. (canceled)
